(19) United States
(12) Patent Application Publication
(10) Pub. No.: US 2026/0014212 A1
(43) Pub. Date: Jan. 15, 2026

(54) INTESTINAL MICROBIOTA PRODUCT, CAPSULE, PREPARATION METHOD AND DEVICE THEREOF, AND USE THEREOF IN PRECISE INTESTINAL MICROBIOTA TRANSPLANTATION

(71) Applicant: Xiamen Treatgut Biotechnology Co., Ltd., Fujian (CN)

(72) Inventors: Wei Xu, Fujian (CN); Bangzhou Zhang, Fujian (CN); Hao Lin, Fujian (CN); Chuanxing Xiao, Fujian (CN); Jianquan He, Fujian (CN); Xiaodie Zhu, Fujian (CN)

(73) Assignee: Xiamen Treatgut Biotechnology Co., Ltd., Fujian (CN)

(21) Appl. No.: 19/264,869

(22) Filed: Jul. 10, 2025

(30) Foreign Application Priority Data
Jul. 10, 2024 (CN) 202410917599.9

(51) Int. Cl. A61K 35/74; A61K 9/48; A61K 47/44; B01J 20/26; B01J 20/28; B01J 47/00; C12N 1/20

(52) U.S. Cl. CPC A61K 35/74; A61K 9/4833; A61K 9/4891; A61K 47/44; B01J 20/26; B01J 20/28004; B01J 20/28085; B01J 47/00; C12N 1/20

(57) ABSTRACT

An intestinal microbiota product, a capsule, a preparation method and device therefore, and use thereof in precise microbiota transplantation are provided, relating to the technical field of intestinal microbiota transplantation. The composition provided by the present disclosure at least includes an eluent and a protective agent. The eluent includes a plant extract and Magnolia officinalis powder at a specific mass ratio, and the protective agent includes a small-molecular polyhydroxy compound, a macromolecular multipolymer, an amine substance, a natural emulsifier and an optional carbonate at a specific mass ratio. The two together constitute an organic whole.

### CROSS-REFERENCE TO RELATED APPLICATION

This application claims the priority benefit of China application serial no. 202410917599.9, filed on Jul. 10, 2024. The entirety of each of the above-mentioned patent applications is hereby incorporated by reference herein and made a part of this specification.

### TECHNICAL FIELD

The present disclosure pertains to the technical field of intestinal microbiota transplantation, and in particular relates to an intestinal microbiota product, a capsule, preparation methods and devices therefor, and use thereof in precise intestinal microbiota transplantation.

### RELATED ART

Intestinal microbiota transplantation refers to the transplantation of a functional microbiota from healthy donor feces into the gastrointestinal tract of a patient to reconstruct a normal intestinal microbiota, thereby achieving the treatment of intestinal and extra-intestinal diseases. As a core technology for targeted reconstruction of the intestinal microbiota, intestinal microbiota transplantation has become an effective strategy for treating diseases such as recurrent Clostridium difficile infection, severe Clostridium difficile infection that does not respond to standard treatment, and overwhelming Clostridium difficile infection that is not suitable for surgery. The cure rate of a single intestinal microbiota transplantation is up to more than 90%. Intestinal microbiota transplantation is a core technology for targeted restoration of gut microbiota. It has emerged as an effective treatment for recurrent and severe Clostridium difficile infections, particularly those unresponsive to standard therapy or unsuitable for surgical intervention. This approach achieves a cure rate exceeding 90% in single-transplant cases. Furthermore, the intestinal microbiota transplantation can also be used as a treatment means for alleviating refractory constipation, treating mild to moderate ulcerative colitis (UC) symptoms, addressing autism, Parkinson's disease, and digestive tract cancers.

Microbiota transplantation is clinically administered to patients primarily via surgical infusion of bacterial solutions or oral capsule delivery. The surgical approach involves microbiota transplantation through methods including nasojejunal intubation, gastroscope instrument channels, percutaneous endoscopic gastrostomy jejunal (PEG-J) tubes, colonoscopy, or enema infusion. This method requires specialized operators and exhibits a high incidence of adverse events, thereby limiting its large-scale clinical implementation. In contrast, oral capsule delivery offers superior application prospects due to enhanced convenience, efficiency, reduced adverse events, and improved patient compliance.

Currently, microbiota transplantation protocols typically encapsulate intestinal microbiota products as freeze-dried powders or microbiota pellets within enteric-coated shells, forming freeze-dried or pellet capsules. The freeze-dried powder is obtained by conducting freeze-drying treatment on a bacterial solution obtained by purification. In addition to the disadvantages of high cost and long cycle, freeze-drying treatment also has a significant killing effect on the bacteria in microbiota pellet products, resulting in a significant reduction in the activity of the microbiota in the capsule and changes in the microbiota structure, reducing the live bacterial load of the freeze-dried capsule, affecting the clinical treatment effect, and even causing side effects in severe cases. In contrast, microbiota pellet is prepared by separating undigested food residues and metabolites from fecal matter, is better preserves microbial viability and community structure compared to freeze-drying.

However, the preparation of microbiota pellet is more complex than that of freeze-dried powder. Improper extraction methods not only cause adverse effects on microbial viability and viable bacterial load in the microbiota pellet, but may also exacerbate undesirable odors. Furthermore, resultant microbiota pellet capsules exhibit limited ambient-temperature stability. When cryogenically stored at −80° C., the moisture in the microbiota pellet will form ice crystals that pierce the bacterial cell walls causing cell death, thereby further affecting the viability and structure of the microbial community. This poses great limitations for clinical deployment.

### SUMMARY OF INVENTION

To achieve microbiota pellet preparation with efficient odor reduction and minimal microbial activity impact, thereby obtaining low-odor, high-viability capsules with enhanced storage stability, the inventors have creatively obtained the technical solution of the present disclosure after extensive and in-depth research.

A first objective of the present disclosure is to provide a composition for extracting a fecal microbiota.

A second objective of the present disclosure is to provide a method for preparing an intestinal microbiota product.

A third objective of the present disclosure is to provide a device for preparing an intestinal microbiota product.

A fourth objective of the present disclosure is to provide an intestinal microbiota product.

A fifth objective of the present disclosure is to provide a method for preparing a capsule.

A sixth objective of the present disclosure is to provide a capsule.

A seventh objective of the present disclosure is to provide use of the aforementioned composition, the device for preparing an intestinal microbiota product, intestinal microbiota product, and/or capsule in precision microbiota transplantation.

Specifically, the fecal microbiota extraction composition provided by the present disclosure includes: an eluent comprising a plant extract and Magnolia officinalis powder in a mass ratio of (0.1-50): (0.001-10), the plant extract is selected from one or more of a Rubus suavissimus extract, a fresh Citrus reticulata extract, a Cymbopogon citratus extract, a clove extract, a bay leaf extract, a charred leaven extract, a licorice extract, a scorched hawthorn fruit extract, an aged Citrus reticulata extract, and a scorched barley sprout extract;
a protective agent comprising a small-molecular polyhydroxy compound, a macromolecular multipolymer, an amine substance, a natural emulsifier and an optional carbonate in a mass ratio of (5-50):(1-55):(1-35):(1-30):(0-15), wherein the small-molecular polyhydroxy compound is selected from one or more of mannitol, maltitol, xylitol, trehalose, stachyose, isomaltulose, manno-oligosaccharide, tea polyphenol, vitamin C and malic acid; the macromolecular multipolymer is selected from one or more of fucoidan, konjac manan, corn starch, ethyl cellulose, acetate starch and agar powder; the amine substance is selected from one or more of a fish collagen peptide, glutathione, polyacrylamide, N-acetyl-D-glucosamine and taurine; the natural emulsifier is selected from one or more of arabic gum, carrageenan, gellan gum, guar gum, xanthan gum and medium-chain triglycerides; and the carbonate is potassium carbonate and/or sodium carbonate; and
an optional separation aid comprising a macroporous resin, a functional resin, and a non-resin adsorbent in a mass ratio of (2-5):(2-6):(2-10).

Further, the preparation of the plant extract specifically includes: taking and subjecting a plant raw material to water extraction treatment to obtain the plant extract.

Further, the plant raw material is selected from one or more of a Rubus suavissimus, a fresh Citrus reticulata, a Cymbopogon citratus, a clove, a bay leaf, a charred leaven, a liquorice root, a scorched hawthorn fruit, an aged Citrus reticulata and a scorched barley sprout.

Further, a method for the water extraction treatment is hot-water leaching and/or ultrasonication.

Further, maltodextrin is added in the water extraction treatment, and a input mass ratio of the plant raw material to the maltodextrin is 1:(1-10).

Further, the plant extract includes the Rubus suavissimus extract, the fresh Citrus reticulata extract, the Cymbopogon citratus extract, and the clove extract in a mass ratio of (1-10):(1-10):(0.1-5):(0.1-2).

Further, the plant extract includes the Rubus suavissimus extract, the fresh Citrus reticulata extract, the Cymbopogon citratus extract, the clove extract and the scorched barley sprout extract in a mass ratio of (5-10):(0.1-5):(1-3):(0.1-2):(0.1-2).

Further, the eluent further includes a bicarbonate that is selected from sodium bicarbonate and/or potassium bicarbonate.

Further, the protective agent is selected from one or more of the following formulas:
formula 1: based on a total mass of the protective agent, the protective agent includes 5-20 wt % of the trehalose, 10-30 wt % of the fucoidan, 15-35 wt % of the fish collagen peptides, 1-10wt % of the vitamin C, 1-5 wt % of the malic acid and 5-20 wt % of the xanthan gum;
formula 2: based on a total mass of the protective agent, the protective agent includes 5-20wt % of the mannitol, 0.1-30 wt % of the arabic gum, 2-10 wt % of the glutathione, 5-30 wt % of the maltitol, 1-30 wt % of the ethyl cellulose and 1-10 wt % of the carrageenan;
formula 3: based on a total mass of the protective agent, the protective agent includes 20-40 wt % of the corn starch, 5-15 wt % of the stachyose, 10-25 wt % of the xylitol, 1-15 wt % of the polyacrylamide, 5-15 wt % of the konjac mannan and 1-10 wt % of the gellan gum; and
formula 4: based on a total mass of the protective agent, the protective agent includes 5-20 wt % of the tea polyphenol, 2-10 wt % of the potassium carbonate, 10-50 wt % of the acetate starch, 2-25 wt % of the N-acetyl-D-glucosamine, 5-30 wt % of the medium-chain triglyceride and 1-5 wt % of the taurine.

Further, the macroporous resin is selected from one or more of epoxy resin, polyester resin, polyvinyl chloride resin, polyethylene resin, polypropylene resin, phenolic resin, polyurethane resin, polystyrene resin, acrylic resin, ether-ketone resin, urea-formaldehyde resin and ketone-formaldehyde resin.

Further, the functional resin is selected from ion-exchange resin and/or chelating resin.

Further, the non-resin adsorbent is selected from one or more of perlite, diatomaceous earth, activated clay, cellulose, titanium dioxide, activated carbon, coconut shell powder and maifan stone.

Further, an average pore diameter of the macroporous resin is 0.4-1.25 mm, an average particle size of the functional resin is 0.5-1 mm, and an average particle size of the non-resin adsorbent is 0.3-1.2 mm.

The method for preparing an intestinal microbiota product using the aforementioned composition provided by this invention includes: step S1: mixing donor feces with the eluent and optional separation aid, and sequentially subjecting to stirring treatment and filtration treatment to obtain a crude microbiota pellet liquid; step S2: taking the crude microbiota pellet liquid and sequentially subjecting to tangential flow filtration treatment and centrifugation treatment to obtain a microbiota pellet; and step S3: taking and mixing the microbiota pellet with the protective agent to obtain the intestinal microbiota product.

Further, in the step S1, a mixing mass ratio of the feces of the donor, the eluent and the separation aid is (5-15):(50-75):(1-3).

Further, in the step S1, the stirring treatment is conducted at a rotation speed of 800-1,500 rpm for a time of 4-15 min.

Further, in the step S1, a pore diameter of a filter membrane in the filtration treatment is 20-2,000 μm.

Further, in the step S2, for the tangential flow filtration treatment, a pore diameter of a filter membrane is 20-2,000 μm, an inlet flow rate is 4-6 L/min/m², an inlet pressure difference is 0.9-1.75 bar, a reflux pressure difference is 0.05-0.2 bar, a transmembrane pressure is 0.5-0.9 bar, and an average membrane flux is 5.1-24.23 L/(m²·h).

Further, in the step S2, the centrifugal treatment is conducted at a rotation speed of 1,000-20,000 g for a time of 2-15 min.

Further, in the step S3, a mixing mass ratio of the microbiota pellet to the protective agent is (5-20):(1-2).

The device for preparing an intestinal microbiota product provided by the present disclosure includes a separation tank, filtering assembly, collection tank I, tangential flow filter, centrifuge and collection tank II fluidically connected in series.

The intestinal microbiota product provided by the present disclosure is prepared by the method for preparing an intestinal microbiota product described above.

The method for preparing a capsule provided by the present disclosure includes: taking a hydrophobic coating material to conduct coating on a capsule shell to obtain a hydrophobic capsule shell; and taking the hydrophobic capsule shell to package the aforementioned intestinal microbiota product to obtain the capsule.

The capsule provided by the present disclosure is prepared by the method for preparing a capsule described above.

The present disclosure further provides use of the aforementioned composition, device for preparing an intestinal microbiota product, and intestinal microbiota product and/or capsule in precise intestinal microbiota transplantation.

Beneficial effects:

The composition provided by the present disclosure at least integrates an eluent for fecal purification, a protective agent for preserving microbial viability. The eluent includes the plant extract and the Magnolia officinalis powder at a specific mass ratio, and the protective agent includes the small-molecular polyhydroxy compound, the macromolecular multipolymer, the amine substance, the natural emulsifier and the optional carbonate at a specific mass ratio. The two together constitute an organic whole, and act synergistically to achieve the simplification of sludge preparation with minimal microbial impact, thereby ultimately obtaining a product exhibiting low odor, high viability, and enhanced storage stability.

It is speculated that the reason why the composition has the aforementioned effects is that: The specific plant extract and Magnolia officinalis powder included in the eluent can effectively adsorb, decompose or transform odor compounds in the feces, thereby achieving the effect of efficient odor removal, and can also mitigate mechanical stress on microbial cells to protect them, thus maintaining the microbiota activity. The protective agent achieves effective coating of the cells in the microbiota pellet through the synergistic action of the small-molecular polyhydroxy compound, the macromolecular multipolymer, the amine substance, the natural emulsifier and the optional carbonate. These components together form cryoprotective matrices preventing ice crystal damage, thereby protecting the cells from injury. They also provide an anaerobic microenvironment for the storage of the microbiota pellet, and the microenvironment can synergize with the residual eluent to preserve taxonomic abundance during cryopreservation. Therefore, the microbiota abundance of the obtained product approximates healthy donor profiles, which facilitates precision transplantation.

In some specific embodiments, when the composition preferably includes the eluent, the protective agent and the separation aid at the same time. The macroporous resin, the functional resin and the non-resin adsorbent in the separation aid cooperatively adsorb particulate and molecular impurities, and under the protection of the eluent. The separation aid can generate controlled shear forces to better lyse fecal matter and increase viable cell yield, and well adsorb non-bacterial micro-impurities such as mucus, bile pigments and mucin in the feces, thereby increasing sludge purity and unit bacterial density without compromising viability.

In some specific embodiments, when the eluent preferably includes the Rubus suavissimus extract, the fresh Citrus reticulata extract, the Cymbopogon citratus extract, the clove extract and the scorched barley sprout extract in a mass ratio of (5-10):(0.1-5):(1-3):(0.1-2):(0.1 -2), it can achieve a more ideal synergistic effect with the protective agent, thereby giving the finally obtained intestinal microbiota product superior storage stability.

### BRIEF DESCRIPTION OF DRAWINGS

FIG. 1 is a schematic structural diagram of a device for preparing an intestinal microbiota product provided in Example 1 of the present disclosure;

FIG. 2 is one of experimental result diagrams of a taxonomic abundance test of an intestinal microbiota product provided in a test example of the present disclosure (Example 2);

FIG. 3 is a second one of experimental result diagrams of the taxonomic abundance test of the intestinal microbiota product provided in the test example of the present disclosure (Comparative Example 2); and

FIG. 4 is a third one of experimental result diagrams of the taxonomic abundance test of the intestinal microbiota product provided in the test example of the present disclosure (Comparative Example 3).

Reference numerals: 1. separation tank; 2. filtering assembly; 3. collection tank I; 4. tangential flow filter; 5. centrifuge; 6. collection tank II.

### DESCRIPTION OF EMBODIMENTS

The composition for extracting a fecal microbiota provided by this invention includes an eluent, a protective agent and an optional separation aid. The eluent removes odorous substances from donor feces, protects cells against mechanical stress damage during processing, and synergizes with the protective agent to enhance the storage stability of the microbiota pellet. The protective agent forms an effective coating around cells within the pellet, protecting them against damage from ice crystals during cryopreservation while providing an anaerobic storage environment. Acting synergistically with residual eluent in the pellet, the protective agent maintains microbiota abundance during low-temperature storage. The separation aid, used in conjunction with the eluent, disrupts donor feces and adsorbs impurity molecules. This facilitates complete cell liberation without compromising microbiota viability, thereby improving pellet quality and bacterial yield per unit mass.

In the present disclosure, the eluent specifically includes a plant extract and Magnolia officinalis powder, wherein a mass ratio of the plant extract to the Magnolia officinalis powder is (0.1-50):(0.001-10), eg. 0.1:0.001, 1:5, 10:8, 23:10, 31:5, 40:9, 50:7 or any value there between.

More specifically, the plant extract is specifically selected from one or more of a Rubus Suavissimus extract, a fresh Citrus reticulata extract, a Cymbopogon citratus extract, a clove extract, a bay leaf extract, a charred leaven extract, a licorice extract, a scorched hawthorn fruit extract, an aged Citrus reticulata extract and a scorched barley sprout extract. At this point, the combined action of one or more of the aforementioned plant extracts and the Magnolia officinalis powder effectively adsorbs and removes odorous substances. When blended at specific ratios, these plant extracts protect cells against mechanical stress while their active constituents promote the growth and reproduction of bacterial, and can continue to play a role during the storage stage of the intestinal microbiota product, so as to achieve the effect of improving the activity of the intestinal microbiota product and maintaining the microbiota taxonomic abundance of the intestinal microbiota product, and give the prepared intestinal microbiota product superior storage stability.

In the present disclosure, specific examples of the method for preparing the plant extract may be, but are not limited to: one or more of water extraction, ethanol extraction, carbon dioxide extraction, glycerol extraction and low-temperature solvent extraction. The aforementioned preparation methods are conventional technical means in the art. Specific processes and conditions can be selected by those skilled in the art as needed, and the present disclosure does not impose any particular limitation thereto.

In the present disclosure, the method for preparing the plant extract is preferably the water extraction, which specifically includes: taking and subjecting a plant raw material to water extraction treatment to obtain the plant extract. At this point, the obtained plant extract is a water-based extract, and the extracted active ingredients are more conducive to the growth and reproduction of bacteria, and its protective effect is more significant in the storage stage of the intestinal microbiota product, thereby giving the prepared intestinal microbiota product superior storage stability. It can play a better protective role in the storage stage of intestinal flora products.

In some specific embodiments, the plant raw material is adaptively selected according to the plant extract to be obtained, and specific examples include: one or more of Rubus suavissimus, fresh Citrus reticulata, Cymbopogon citratus, clove, a bay leaf, charred leaven, liquorice root, a scorched hawthorn fruit, aged Citrus reticulata and a scorched barley sprout; and each plant extract can be prepared by mixing multiple plant raw materials together, or can be prepared independently.

In some specific embodiments, specific examples of the water extraction treatment method include, but are not limited to: hot-water leaching and/or ultrasonication. The aforementioned hot-water leaching and ultrasonication are conventional technical means in the art. Specific processes and conditions can be selected by those skilled in the art as needed, and the present disclosure does not impose any special limitation thereto.

In some specific embodiments, maltodextrin is preferably added into the water extraction treatment, and an input mass ratio of the plant raw material to the maltodextrin is preferably 1:(1-10), e.g. 1:1, 1:1.5, 1:2.3, 1:4, 1:6, 1:8.7, 1:9, 1:10 or any value there between. At this point, the maltodextrin can increase the viscosity and dispersibility of the water extraction system, which effectively removes unexpected impurity molecules while improving the extraction rate of the active ingredients, so as to achieve a better extraction effect.

In the present disclosure, the plant extract in the eluent preferably includes the Rubus Suavissimus extract, the fresh Citrus reticulata extract, the Cymbopogon citratus extract and the clove extract in a mass ratio of (1-10):(1-10):(0.1-5):(0.1-2), e.g. 1:1:0.1:0.1, 1:3:2:2, 1:10:5:2, 2.5:5.5:0.75:1, 3:1:0.1:2, 3.5:4:1.2:0.55, 4:2:2:1.5, 5.5:2.5:1:0.7, 6:2:1.15:0.5 or any value there between. At this point, the obtained eluent can achieve an ideal synergistic effect with the protective agent, thereby giving the final intestinal microbiota product superior storage stability.

In the present disclosure, the plant extract in the eluent preferably includes the Rubus suavissimus extract, the fresh Citrus reticulata extract, the Cymbopogon citratus extract, the clove extract and the scorched barley sprout extract in a mass ratio of (5-10):(0.1-5):(1-3):(0.1-2):(0.1-2), e.g. 5:0.1:1:0.1:2, 8:3:2:2:0.1, 6:2:1.15:0.5:0.5 or any value there between. At this point, the obtained eluent can achieve an ideal synergistic effect with the protective agent, thereby giving the final intestinal microbiota product superior storage stability.

In the present disclosure, the eluent preferably further includes a bicarbonate, and specific examples of the bicarbonate may be but are not limited to: sodium bicarbonate and/or potassium bicarbonate. At this point, the bicarbonate can change the electrical properties of the solution during the treatment of the feces of the donor, thereby achieving better adsorption and removal effects on the odorous substances.

In some specific embodiments, based on the total mass of the eluent, the concentration of the bicarbonate is preferably 0.5-10 wt %, e.g. 0.5 wt %, 0.8 wt %, 1 wt %, 2.4 wt %, 5.5 wt %, 8 wt %, 10 wt % or any value there between. At this point, the addition of the bicarbonate can greatly improve the odor removal effect and is beneficial to improving the microbiota activity and storage stability of the microbiota pellet.

In the present disclosure, the protective agent specifically includes a small-molecular polyhydroxy compound, a macromolecular multipolymer, an amine substance, a natural emulsifier and an optional carbonate, and the mass ratio of the small-molecular polyhydroxy compound, the macromolecular polymer, the amine substance, the natural emulsifier and the optional carbonate is specifically (5-50):(1-55):(1-35):(1-30):(0-15), e.g. 5:1:1:1, 10:20:15:27:1, 50:55:28:17:13 or any value there between.

More specifically, the small-molecular polyhydroxy compound is an organic small-

molecule compound containing multiple hydroxyl groups in its molecular structure, which has the effects of anti-oxidation and maintaining a relatively good state of the intestinal microbiota product. Specific examples include: one or more of mannitol, maltitol, xylitol, trehalose, stachyose, isomaltulose, manno-oligosaccharide, tea polyphenols, vitamin C and malic acid. The macromolecular polymer is a kind of high-molecular substances with a certain viscosity and high biological safety after being dissolved in water, which can effectively encapsulate the microbiota and provide an anaerobic microenvironment suitable for the survival of the microbiota. Specific examples include: one or more of fucoidan, konjac mannan, corn starch, ethyl cellulose, acetate starch and agar powder. The amine substance is a product obtained by substituting one or more hydrogen atoms in an ammonia molecule with a hydrocarbyl group. The addition of the amine substance is beneficial to the growth and reproduction of the microbiota. Specific examples include: one or more of a fish collagen peptides, glutathione, polyacrylamide, N-acetyl-D-glucosamine and taurine. The natural emulsifier is an emulsifier composed of natural raw materials, which can improve the dispersibility of other substances to achieve a better protective effect on the intestinal microbiota product. Specific examples include: one or more of arabic gum, carrageenan, gellan gum, guar gum, xanthan gum and medium-chain triglycerides (MCTs). Specific examples of the carbonate include, but are not limited to: potassium carbonate and/or sodium carbonate. At this point, the synergistic cooperation of the small-molecular polyhydroxy compound, the macromolecular polymer, the amine substance, the natural emulsifier and the optional carbonate in the protective agent can enable protection of the intestinal microbiota product from two aspects of reducing the physical damage to the microbiota and improving the good state of the microbiota, so that the intestinal microbiota product can still maintain a high microbiota activity when stored for a long time at −80° C., has relatively small changes in the microbiota taxonomic abundance, and has superior storage stability.

In some specific embodiments, based on the total mass of the protective agent, the protective agent includes: 5-20 wt % of the trehalose, e.g. 5 wt %, 8 wt %, 10 wt %, 15 wt %, 20 wt % or any value there between; 10-30 wt % of the fucoidan, e.g. 10 wt %, 12.5 wt %, 15 wt %, 18 wt %, 20 wt %, 30 wt % or any value there between; 15-35 wt % of the fish collagen peptides, e.g. 15 wt %, 16 wt %, 18 wt %, 20 wt %, 25 wt %, 30 wt %, 35 wt % or any value there between; 1-10 wt % of the vitamin C, e.g. 1 wt %, 3 wt %, 7 wt %, 10 wt % or any value there between; 1-5 wt % of the malic acid, e.g. 1wt %, 1.5 wt %, 2 wt %, 3.5 wt %, 4 wt %, 5 wt % or any value there between; and 5-20 wt % of the xanthan gum, e.g. 5 wt %, 8 wt %, 10 wt %, 15 wt %, 20 wt % or any value there between.

In some specific embodiments, based on the total mass of the protective agent, the protective agent includes: 5-20 wt % of the mannitol, e.g. 5 wt %, 7.5 wt %, 10 wt %, 15 wt %, 18 wt %, 20 wt % or any value there between; 0.1-30 wt % of the arabic gum, e.g. 0.1 wt %, 1 wt %, 5 wt %, 8 wt %, 10 wt %, 13 wt %, 18 wt %, 20 wt %, 25 wt %, 30 wt % or any value there between; 2-10wt % of the glutathione, e.g. 2 wt %, 2.5 wt %, 3 wt %, 4 wt %, 8 wt %, 10 wt % or any value there between; 5-30 wt % of the maltitol, e.g. 5 wt %, 6.5 wt %, 10 wt %, 12 wt %, 15 wt %, 20 wt %, 25 wt %, 30 wt % or any value there between; 1-30 wt % of the ethyl cellulose, e.g. 1 wt %, 3 wt %, 5 wt %, 10 wt %, 20 wt %, 30 wt % or any value there between; and 1-10 wt % of the carrageenan, e.g. 1 wt %, 1.5 wt %, 2 wt %, 3.5 wt %, 5 wt %, 8 wt %, 10 wt % or any value there between.

In some specific embodiments, based on the total mass of the protective agent, the protective agent includes: 20-40 wt % of the corn starch, e.g. 20 wt %, 21 wt %, 23 wt %, 28 wt %, 30 wt %, 31 wt %, 38 wt %, 40 wt % or any value there between; 5-15 wt % of the stachyose, e.g. 5 wt %, 8 wt %, 10 wt %, 12 wt %, 15 wt % or any value there between; 10-25 wt % of the xylitol, e.g. 10 wt %, 15 wt %, 20 wt %, 25 wt % or any value there between; 1-15 wt % of the polyacrylamide, e.g. 1 wt %, 3 wt %, 5 wt %, 7 wt %, 9 wt %, 15 wt % or any value there between; 5-15 wt % of the konjac mannan, e.g. 5 wt %, 7 wt %, 9 wt %, 10 wt %, 12 wt %, 13 wt %, 15 wt % or any value there between; and 1-10 wt % of the gellan gum, e.g. 1 wt %, 2.5 wt %, 5 wt %, 7 wt %, 8 wt %, 9 wt %, 10 wt % or any value there between.

In some specific embodiments, based on the total mass of the protective agent, the protective agent includes: 5-20 wt % of the tea polyphenol, e.g. 5 wt %, 8 wt %, 9 wt %, 10 wt %, 15 wt %, 20 wt % or any value there between; 2-10 wt % of the potassium carbonate, e.g. 2 wt %, 2.5 wt %, 5 wt %, 7.5 wt %, 10 wt % or any value there between; 10-50 wt % of the acetate starch, e.g. 10 wt %, 12 wt %, 15 wt %, 20 wt %, 28 wt %, 37 wt %, 40 wt %, 45 wt %, 50 wt % or any value there between; 2-25 wt % of the N-acetyl-D-glucosamine, e.g. 2 wt %, 4.5 wt %, 5 wt %, 8 wt %, 10 wt %, 12 wt %, 18 wt %, 20 wt %, 25 wt % or any value there between; 5-30 wt % of the medium-chain triglycerides, e.g. 5 wt %, 8 wt %, 10 wt %, 12 wt %, 18 wt %, 20 wt %, 25 wt %, 30 wt % or any value there between; and 1-5 wt % of the taurine, e.g. 1 wt %, 2 wt %, 3.8 wt %, 4 wt %, 5 wt % or any value there between.

In the present disclosure, the separation aid specifically includes a macroporous resin, a functional resin and a non-resin adsorbent, and the mass ratio of the macroporous resin, the functional resin and the non-resin adsorbent is (2-5):(2-6):(2-10), e.g. 1:1:1, 1:3:2, 3:2:2, 3:6:5, 4:5:9, 5:6:10 or any value there between.

More specifically, the macroporous resin is a kind of organic macromolecular polymer adsorbents having a three-dimensional spatial stereoscopic pore structure internally, which can effectively adsorb odor substances and other impurity molecules in the feces of the donor. Specific examples include but are not limited to: one or more of epoxy resin, polyester resin, polyvinyl chloride resin, polyethylene resin, polypropylene resin, phenolic resin, polyurethane resin, polystyrene resin, acrylic resin, ether-ketone resin, urea-formaldehyde resin and ketone-formaldehyde resin. The functional resin is a type of resin that has specific functional groups in its macromolecular chain, which can achieve adsorption of odorous substances and other impurity molecules through electrostatic forces and/or complexation. Specific examples include but are not limited to: one or more of cation exchange resin, anion exchange resin and chelating resin. The non-resin adsorbent can also provide a secondary shear force to break up the feces of the donor, while having the ability of effectively adsorbing odorous substances and other impurity molecules in the feces, which is beneficial to the free of the microbiota. Specific examples include but are not limited to: one or more of perlite, diatomaceous earth, activated clay, cellulose, titanium dioxide, activated carbon, coconut shell powder and maifan stone.

In some specific embodiments, an average pore diameter of the macroporous resin is preferably 0.4-1.25 mm, e.g. 0.4 mm, 0.5 mm, 0.63 mm, 0.9 mm, 1 mm, 1.15 mm, 1.2 mm, 1.25 mm or any value there between; an average particle size of the functional resin is preferably 0.5-1 mm, e.g. 0.5 mm, 0.55 mm, 0.58 mm, 0.6 mm, 0.67 mm, 0.8 mm, 0.9 mm, 1 mm or any value there between; and an average particle size of the non-resin adsorbent is preferably 0.3-1.2 mm, e.g. 0.3 mm, 0.38 mm, 0.4 mm, 0.6 mm, 0.9 mm, 1 mm, 1.2 mm or any value there between. At this point, the macroporous resin, the functional resin and the functional resin can cooperate better and act together with the eluent to achieve a better deodorizing effect.

In the method for preparing an intestinal microbiota product provided by this invention, the feces of a donor is treated with the aforementioned eluent, protective agent and optional separation aid, specifically including: step S1: mixing donor feces with the eluent and optionally the separation aid, and sequentially subjecting to stirring treatment and filtration treatment to obtain a crude microbiota pellet liquid; step S2: taking the crude microbiota pellet liquid and sequentially subjecting to tangential flow filtration treatment and centrifugation treatment to obtain a microbiota pellet; and step S3: taking and mixing the microbiota pellet with the protective agent to obtain the intestinal microbiota product.

In the present disclosure, in the step S1, a mixing mass ratio of the feces of the donor, the eluent and the separation aid is preferably (5-15):(50-75):(1-3), e.g. 5:50:1, 10:63:1, 13:75:3, 15:50:3, 15:74:3 or any value there between. At this point, odors can be removed more efficiently with less negative impact on microbiota activity, ensuring that they can maintain a high activity state in subsequent applications.

In some specific embodiments, the feces of the donor, the eluent and the separation aid can be mixed by simultaneous addition and/or addition in batches. The simultaneous addition specifically refers to: directly mixing the feces of the donor, the separation aid and all the eluent, and then subjecting to stirring treatment and filtration treatment; and the addition in batches specifically refers to: dividing the eluent into multiple portions, and mixing with the feces of the donor and the separation aid in sequence, where stirring is performed after each time of mixing, and filtration treatment is performed only after all of the eluent has been added. In some preferred embodiments, the feces of the donor, the eluent and the separation aid are preferably mixed by addition in batches, which can reduce the impact of the mixing and the stirring treatment processes on the activity of the microbiota.

In the present disclosure, in the step S1, the conditions for the stirring treatment include a rotation speed of preferably 800-1,500 rpm, e.g. 800 rpm, 850 rpm, 900 rpm, 1,000 rpm, 1,200 rpm, 1,500 rpm or any value there between; and a time of preferably 4-15 min, e.g. 4 min, 5 min, 8 min, 10 min, 12 min, 15 min or any value there between. At this point, the aforementioned preferred conditions for stirring treatment are adopted to reduce the adverse effects on the activity of the microbiota during the stirring treatment, thereby achieving the effect of improving the activity of the microbiota in the microbiota pellet.

In this invention, in the step S1, a pore diameter of a filter membrane in the filtration treatment is preferably 20-2,000 μm, e.g. 20 μm, 25 μm, 50 μm, 100 μm, 237 μm, 500 μm, 750 μm, 1,000 μm, 1,500 μm, 2,000 μm or any value there between. At this point, the intestinal microbiota can be effectively separated from unexpected impurities to achieve a better purification effect.

In the present disclosure, in the step S2, during the tangential flow filtration treatment, the intestinal microbiota in the crude microbiota pellet liquid is collected through the filter membrane, and the impurity particles are effectively retained on the surface of the filter membrane, thereby achieving efficient and gentle separation and collection of the intestinal microbiota, and improving the quality and the amount of bacterial yield per unit mass of the recovered microbiota pellet.

In some specific embodiments, the conditions for the tangential flow filtration treatment include a pore diameter of the filter membrane of preferably 20-2,000 μm, e.g. 20 μm, 30 μm, 50 μm, 150 μm, 250 μm, 1,000 μm, 1,500 μm, 2,000 μm or any value there between; an inlet flow rate of preferably 4-6 L/min/m², e.g. 4 L/min/m², 4.5 L/min/m², 5 L/min/m², 5.8 L/min/m², 6 L/min/m² or any value there between; an inlet pressure difference of preferably 0.9-1.75 bar, e.g. 0.9 bar, 0.95 bar, 1 bar, 1.23 bar, 1.57 bar, 1.6 bar, 1.75 bar or any value there between; a reflux pressure difference of preferably 0.05-0.2 bar, e.g. 0.05 bar, 0.08 bar, 0.1 bar, 0.13 bar, 0.15 bar, 0.18 bar, 0.2 bar or any value there between; a transmembrane pressure of preferably 0.5-0.9 bar, e.g. 0.5 bar, 0.55 bar, 0.6 bar, 0.63 bar, 0.68 bar, 0.7 bar, 0.85 bar, 0.9 bar or any value there between; and an average membrane flux of preferably 5.1-24.23 L/(m²·h, e.g. 5.1 L/(m²·h, 5.3 L/(m²·h, 6 L/(m²·h, 7.8 L/(m²·h, 10 L/(m²·h, 11.5 L/(m²·h, 15 L/(m²·h, 18.6 L/(m²·h, 20 L/(m²·h, 23.1 L/(m²·h, 24.23 L/(m²·h or any value there between.

In some specific embodiments, the conditions for the centrifugal treatment include a rotation speed of preferably 1,000-20,000 g, e.g. 1,000 g, 1,001 g, 1,500 g, 1,800 g, 2,000 g, 5,000 g, 10,000 g, 20,000 g or any value there between; and a time of 2-15 min, e.g. 2 min, 2.8 min, 3 min, 5 min, 9 min, 10 min, 12 min, 15 min or any value there between.

In the present disclosure, in the step S3, the mixing mass ratio of the microbiota pellet to the protective agent is preferably (5-20):(1-2), e.g. 5:1, 8:1, 10:1, 20:1, 7:1.5, 17:1.8, 5:2, 19:2 or any value there between. At this point, taking and mixing the microbiota pellet with the protective agent according to the aforementioned mixing mass ratio can better achieve the protective effect of the protective agent on the microbiota in the intestinal microbiota product.

The device for preparing an intestinal microbiota product provided by this invention is obtained by designing based on the method for preparing an intestinal microbiota product described above, and in order to reduce the impact on the microbiota, the device is operated in a closed state. The device specifically includes: a separation tank, a filtering assembly, a collection tank I, a tangential flow filter, a centrifuge and a collection tank II which are fluidically connected in series.

In the present disclosure, the filtering assembly, the tangential flow filter and the centrifuge are a type of devices commonly used in the prior art, as long as they can achieve filtration, tangential flow filtration or centrifugation functions, and this invention does not impose any special limitation on their specific structures.

The present disclosure further provides an intestinal microbiota product prepared by the method for preparing an intestinal microbiota product as described above, where the intestinal microbiota product has a microbiota activity higher than 70%, and after being stored at −80° C. for 360 d, the microbiota activity of the intestinal microbiota product is still maintained at more than 53%. The intestinal microbiota product has high microbiota activity and superior storage stability.

The method for preparing the capsule provided by the present disclosure specifically includes: taking a hydrophobic coating material to conduct coating on a capsule shell to obtain a hydrophobic capsule shell; and taking the hydrophobic capsule shell to package the aforementioned intestinal microbiota product to obtain the capsule. The hydrophobic coating material includes beeswax, mineral oil, vegetable oil and emulsifier, and a mass ratio of the beeswax, the mineral oil, the vegetable oil and the emulsifier is (5-10):(1-3):(3-5):(1-3), e.g. 5:1:3:1, 4:3:5:2, 6:1:3:3, 8:2:5:3, 10:3:5:3 or any value there between. At this point, the prepared hydrophobic capsule shell includes a hydrophobic outer shell and a hydrophobic coating inner layer covering the inner and outer faces of the capsule shell. The hydrophobic outer shell has excellent resistance to gastric acid erosion and can maintain structural integrity in the gastric environment, thereby avoiding premature dissolution; and the hydrophobic coating inner layer can provide a better protective environment for the microbiota, and the synergistic action of the outer shell and the coating inner layer can significantly enhance the colonization effect of the microbiota in the intestine.

In some specific embodiments, specific examples of the vegetable oil may be, but are not limited to: one or more of soybean oil, sunflower oil and olive oil; and specific examples of the emulsifier include but are not limited to: glyceryl fatty acid ester and/or soy lecithin.

In the present disclosure, the coating manner may be specifically one or more of immersion, spray coating and spin coating, as long as the hydrophobic coating material can be coated on the inner and outer faces of the capsule shell. The present disclosure does not impose any special limitation on its specific process and conditions.

The capsule provided in the present disclosure is prepared by the aforementioned method for preparing a capsule.

The present disclosure further provides use of the aforementioned composition, device for preparing an intestinal microbiota product, intestinal microbiota product and/or capsule in precise intestinal microbiota transplantation.

Hereinafter, embodiments of the present disclosure will be described in detail, and examples of the embodiments are intended to explain the present disclosure and should not be construed as limiting the present disclosure. If no specific technology or condition is indicated in the examples, it shall be carried out according to the technology or condition described in the literature in the art or according to product instructions. All of the used agents or instruments which are not specified with the manufacturer are conventional commercially-available products.

The raw materials used in the following preparation examples and examples and sources thereof are specifically:

fucoidan (Qingdao Bright Moon Seaweed Biological Health Technology Group Co., Ltd., Cat. No. 9072-19-9);
fish collagen peptides (Hebei Klondo Biotechnology Co., Ltd., Cat. No. 472-61-5);
xanthan gum (Guangzhou Yuanchang Trade Co., Ltd., Cat. No. 11138-66-2);
arabic gum (Jiangsu Caiwei Biotechnology Co., Ltd., Cat. No. 9000 Jan. 5);
ethyl cellulose (Shanghai Zhongfeng Biotechnology Co., Ltd., Cat. No. 618-384-9);
carrageenan (Chongqing Tianrun Biological Products Co., Ltd., Cat. No. 11114-20-8);
polyacrylamide (Henan Shuifangcheng Water Purifying Materials Co., Ltd., Cat. No. 9003 May 8);
konjac manan (Xi'an Lavia Biotechnology Co., LTD., Cat. No. 37220-17-0);
gellan gum (Chongqing Tianrun Biological Products Co., Ltd., Cat. No. 71010-52-1);
acetate starch (Chongqing Tianrun Biological Products Co., Ltd., Cat. No. 9045-28-7);
medium-chain triglycerides (MCTs) (Shandong Pingju Biotechnology Co., Ltd., Cat. No. 538-24-9);
macroporous aromatic polymer resin (Shanghai yuanye Bio-Technology Co., Ltd., Cat. No. S14162);
ion-exchange resin (Shanghai yuanye Bio-Technology Co., Ltd., Cat. No. 63181-94-2).

### Preparation Example 1

This preparation example was used for illustrating the preparation of an eluent, which specifically included:
step S1. 50 g of Rubus suavissimus was taken and mixed with 1,000 mL of distilled water, soaked for 10 min, then heated to boiling, treated at 100°° C. for 20 min, and filtered, and the filtrate was collected, added with maltodextrin at an addition amount of 10% (v/v), and freeze-dried under conditions of −40° C. and 0.05 MPa to obtain a Rubus suavissimus extract;
step S2. 50 g of Cymbopogon citratus was taken and mixed with 1,000 mL of distilled water, soaked for 10 min, then heated to boiling, treated at 100° C. for 20 min, and filtered, and the filtrate was collected, added with maltodextrin at an addition amount of 10% (v/v), and freeze-dried under conditions of −40°° C. and 0.05 MPa to obtain a Cymbopogon citratus extract;
step S3. 50 g of clove was taken and mixed with 1,000 mL of distilled water, soaked for 10 min, then heated to boiling, treated at 100° C. for 20 min, and filtered, and the filtrate was collected, added with maltodextrin at an addition amount of 10% (v/v), and freeze-dried under conditions of −40°° C. and 0.05 MPa to obtain a clove extract;
step S4. 50 g of fresh Citrus reticulata was crushed, then added with 100 mL of distilled water, subjected to ultrasonic-assisted extraction at room temperature for 20 min, and filtered, and the filtrate was collected, subjected to distillation at reduced pressure under conditions of at −40° C. and 0.05 MPa to obtain a pasty fresh Citrus reticulata extract;
step S5. 10 g of Magnolia officinalis was taken, washed, oven dried, and crushed, then passed through a 50-mesh sieve to obtain Magnolia officinalis powder; and
step S6. The Rubus suavissimus extract, the fresh Citrus reticulata extract, the Cymbopogon citratus extract, the clove extract and the Magnolia officinalis powder were taken in a mass ratio of 6:2:1.15:0.5:0.35, and mixed uniformly to obtain an eluent.

### Preparation Example 2

This preparation example adopted the method provided in Preparation Example 1 to prepare an eluent, except that in the step S6, the mass ratio of the Rubus suavissimus extract, the fresh Citrus reticulata extract, the Cymbopogon citratus extract, the clove extract and the Magnolia officinalis powder was 5.5:2.5:1:0.7:0.35, with the other conditions being the same, so as to obtain the eluent.

### Preparation Example 3

This preparation example adopted the method provided in Preparation Example 1 to prepare an eluent, except that in the step S6, the mass ratio of the Rubus Suavissimus S. Lee extract, the fresh Citrus reticulata extract, the Cymbopogon citratus extract, the clove extract and the Magnolia officinalis powder was 2.5:5.5:0.75:1:0.25, with the other conditions being the same, so as to obtain the eluent.

### Preparation Example 4

This preparation example adopted the method provided in Preparation Example 1 to prepare an eluent, except that in the step S6, the mass ratio of the Rubus suavissimus extract, the fresh Citrus reticulata extract, the Cymbopogon citratus extract, the clove extract and the Magnolia officinalis powder was 3.5:4:1.2:0.55:0.75, with the other conditions being the same, so as to obtain the eluent.

### Preparation Example 5

This preparation example adopted the method provided in Preparation Example 1 to prepare an eluent, except that in the step S6, the mass ratio of the Rubus suavissimus extract, the fresh Citrus reticulata extract, the Cymbopogon citratus extract, the clove extract and the Magnolia officinalis powder was 4:2:2:1.5:0.5, with the other conditions being the same, so as to obtain the eluent.

### Preparation Example 6

This preparation example adopted the method provided in Preparation Example 1 to prepare an eluent, except that a scorched barley sprout extract was further added in the step S6, and the mass ratio of the Rubus suavissimus extract, the fresh Citrus reticulata extract, the Cymbopogon citratus extract, the clove extract, the scorched barley sprout extract and the Magnolia officinalis powder was 6:2:1.15:0.5:0.5:0.35, with the other conditions being the same, so as to obtain the eluent.

In this preparation example, the preparation of the scorched barley sprout extract specifically included: 50 g of scorched barley sprout was taken and mixed with 1,000 mL of distilled water, soaked for 10 min, then heated to boiling, treated at 100° C. for 20 min, and filtered, and the filtrate was collected, added with maltodextrin at an addition amount of 10% (v/v), and freeze-dried under conditions of-40° C. and 0.05 MPa to obtain the scorched barley sprout extract.

### Preparation Example 7

This preparation example adopted the method provided in Preparation Example 1 to prepare an eluent, except that in the step S6, the fresh Citrus reticulata extract was replaced with an equal parts by mass of an aged Citrus reticulata extract, with the other conditions being the same, so as to obtain the eluent.

In this preparation example, the preparation of the aged Citrus reticulata extract specifically included: 50 g of aged Citrus reticulata was taken and mixed with 1,000 mL of distilled water, soaked for 10 min, then heated to boiling, treated at 100°° C. for 20 min, and filtered, and the filtrate was collected, added with maltodextrin at an addition amount of 10% (v/v), and freeze-dried under conditions of −40° C. and 0.05 MPa to obtain the aged Citrus reticulata extract.

### Preparation Example 8

This preparation example adopted the method provided in Preparation Example 1 to prepare an eluent, except that a sodium hydrogencarbonate was further added in the step S6, and the mass ratio of the Rubus suavissimus extract, the fresh Citrus reticulata extract, the Cymbopogon citratus extract, the clove extract, the sodium hydrogencarbonate and the Magnolia officinalis powder was 6:2:1.15:0.5:0.5:0.35, with the other conditions being the same, so as to obtain the eluent.

### Comparative Preparation Example 1

This comparative preparation example adopted the method provided in Preparation Example 1 to prepare an eluent, except that in the step S6, the Rubus suavissimus extract, the fresh Citrus reticulata extract, the Cymbopogon citratus extract and the clove extract were replaced with equal parts by weight of an Artemisia argyi extract, that was, the mass ratio of the Artemisia argyi extract to the Magnolia officinalis powder was 9.65:0.35, with the other conditions being the same, so as to obtain the eluent.

In this comparative preparation example, the preparation of the Artemisia argyi extract specifically included: 50 g of Artemisia argyi was taken and mixed with 1,000 mL of distilled water, soaked for 10 min, then heated to boiling, treated at 100°° C. for 20 min, and filtered, and the filtrate was collected, added with maltodextrin at an addition amount of 10% (v/v), and freeze-dried under conditions of −40° C. and 0.05 MPa to obtain the Artemisia argyi extract.

### Preparation Example 9

This preparation example provided preparation of a protective agent, which specifically included: 20 parts by mass of trehalose, 30 parts by mass of fucoidan, 20 parts by mass of fish collagen peptides, 8 parts by mass of vitamin C, 12 parts by mass of malic acid and 10 parts by mass of xanthan gum were taken and mixed uniformly to obtain the protective agent.

### Preparation Example 10

This preparation example provided preparation of a protective agent, which specifically included: 15 parts by mass of mannitol, 25 parts by mass of arabic gum, 10 parts by mass of glutathione, 20 parts by mass of maltitol, 25 parts by mass of ethyl cellulose and 5 parts by mass of carrageenan were taken and mixed uniformly to obtain the protective agent.

### Preparation Example 11

This preparation example provided preparation of a protective agent, which specifically included: 35 parts by mass of corn starch, 15 parts by mass of stachyose, 25 parts by mass of xylitol, 5 parts by mass of polyacrylamide, 15 parts by mass of konjac mannan and 5 parts by mass of gellan gum were taken and mixed uniformly to obtain the protective agent.

### Preparation Example 12

This preparation example provided preparation of a protective agent, which specifically included: 20 parts by mass of tea polyphenol, 10 parts by mass of potassium carbonate, 35 parts by mass of acetate starch, 10 parts by mass of N-acetyl-D-glucosamine, 20 parts by mass of medium-chain triglycerides (MCTs) and 5 parts by mass of taurine were taken and mixed uniformly to obtain the protective agent.

### Comparative Preparation Example 2

This comparative preparation example adopted the method provided in Preparation Example 9 to prepare a protective agent, except that the xanthan gum was replaced with an equal parts by mass of fucoidan, with the other conditions being the same, so as to obtain the protective agent.

### Comparative Preparation Example 3

This comparative preparation example adopted the method provided in Preparation Example 9 to prepare a protective agent, except that the marine collagen peptides was replaced with an equal parts by mass of fucoidan, with the other conditions being the same, so as to obtain the protective agent.

It should be noted that the feces of a donor used in the following examples were the same.

### Example 1

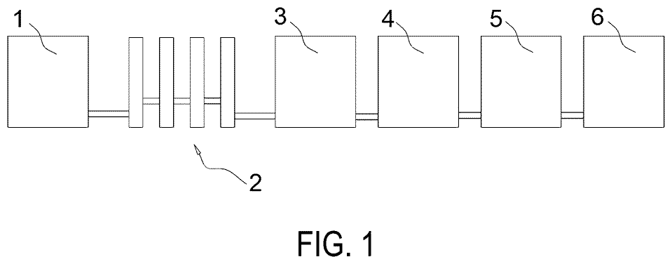

This example was used for illustrating a device for preparing an intestinal microbiota product and a method for preparing an intestinal microbiota product. Referring to FIG. 1, the device specifically included a separation tank 1, a filtering assembly 2, a collection tank I 3, a tangential flow filter 4, a centrifuge 5 and a collection tank II 6. A stirrer was installed in the separation tank 1, the filtering assembly 2 included first-to-fourth-stage filtering units arranged in series and fluidically connected in series through sterile catheters, and the pore diameter of the filter membrane of the first-to-fourth-stage filtering units were 1,000 μm, 500 μm, 150 μm and 100 μm respectively in sequence. The separation tank 1 was communicated with the first-stage filtering unit through a sterile catheter, the fourth-stage filtering unit was communicated with the collection tank I 3 through a sterile catheter, the collection tank I 3 was communicated with an water inlet of the tangential flow filter 4 through a sterile catheter, an filtering outlet of the tangential flow filter 4 was communicated with the centrifuge 5 through a sterile catheter, a pore diameter of the filter membrane of the tangential flow filter 4 was 60 μm, the outlet of the centrifuge 5 was communicated with the collection tank II6 through a sterile catheter, and a rotation speed of the centrifuge 5 was 5,000 g.

This example provided a method for preparing an intestinal microbiota product using the aforementioned device, which specifically included:
step S1. (1) Screening was conducted according to the existing “Chinese Expert consensus on clinical application management of fecal microbiota transplantation (2022 edition)” to obtain qualified feces of a donor;
(2) the eluent provided in Preparation Example 1 and physiological saline were taken and mixed uniformly in a mass ratio of 1:9 to obtain an eluent; 100 g of the feces of a donor and 500 g of an eluent were taken and added into the separation tank 1, and subjected to stirring treatment at 1,000 rpm for 5 min, and then added with 500 g of the eluent, and continually subjected to stirring treatment under a condition of 1,000 rpm for 5 min to obtain a mixed solution; and
(3) the mixed solution entered the filtering assembly 2 through a sterile catheter, and was subjected to filtration treatment through the first-stage, second-stage, third-stage and fourth-stage filtering units, and the filtrate was collected with the collection tank 13 to obtain a crude microbiota pellet liquid.
step S2. The crude microbiota pellet liquid entered the tangential flow filter 4 through a sterile catheter, and was subjected to tangential flow filtration treatment under the conditions of a feed flow rate of 5 L/min/m², an inlet pressure difference of 1.5 bar, a reflux pressure difference of 0.1 bar, a transmembrane pressure of 0.8 bar and an average membrane flux of 20 L/(m²·h. The microbiota pellet filtrate obtained by the tangential flow filter was collected with the centrifuge 5 and subjected to centrifugation at a rotation speed of 5,000 g for 10 min to obtain a microbiota pellet; and the microbiota pellet obtained by the centrifugation was collected with the collection tank II6.
step S3. The microbiota pellet was taken and mixed with the protective agent provided in Preparation Example 9 in a mass ratio of 10:1 to obtain an intestinal microbiota product.

### Example 2

This example adopted the device and method provided in Example 1 to prepare an intestinal microbiota product, except that 50 g of a separation aid was further added in the step S1 (2), with the other conditions being the same, so as to obtain an intestinal microbiota product.

In this example, the separation aid specifically included a macroporous aromatic polymer resin, an ion-exchange resin and a maifan stone in a mass ratio of 20:30:50.

### Examples 3-12

Examples 3-12 adopted the device and method provided in Example 2 to prepare intestinal microbiota product, except that the eluent added in the step S1 (2) and the protective agent added in the step S3 and the dosage thereof were different, specifically as shown in Table 1, with the other conditions being the same, so as to obtain intestinal microbiota product.

TABLE 1

| | | | Step S3 | |
|---|---|---|---|---|
| | | | | Mass ratio |
| | Step S1 | | | (microbiota |
| Intestinal | | Mass ratio | | pellet:pro- |
| microbiota | | (eluent:physio- | Protective | tective |
| product | Eluent | logical saline) | agent | agent) |
| Example 3 | Preparation | 1:9 | Preparation | 10:1 |
| | Example 1 | | Example 10 | |
| Example 4 | | | Preparation | |
| | | | Example 11 | |
| Example 5 | | | Preparation | |
| | | | Example 12 | |
| Example 6 | Preparation | | Preparation | 10:1 |
| | Example 2 | | Example 9 | |
| Example 7 | Preparation | | | 5:2 |
| | Example 3 | | | |
| Example 8 | Preparation | | | 10:1 |
| | Example 4 | | | |
| Example 9 | Preparation | | | 10:1 |
| | Example 5 | | | |
| Example 10 | Preparation | | | 20:1 |
| | Example 6 | | | |
| Example 11 | Preparation | | | |
| | Example 7 | | | |
| Example 12 | Preparation | | | |
| | Example 8 | | | |

### Comparative Example 1

This comparative example adopted the method provided in Example 2 to prepare an intestinal microbiota product, except that in the step S1 (2), an equal mass of physiological saline was used instead of the eluent to mix with the feces of the donor, with the other conditions being the same, so as to obtain an intestinal microbiota article.

### Comparative Example 2

This comparative example adopted the method provided in Example 2 to prepare an intestinal microbiota product, except that in the step S1 (2), an equal mass of the eluent provided in Comparative Preparation Example 1 was used instead of the eluent provided in Preparation Example 1, with the other conditions being the same, so as to obtain an intestinal microbiota product.

### Comparative Example 3

This comparative example adopted the method provided in Example 2 to prepare an intestinal microbiota product, except that in the step S3, an equal mass of the protective agent provided in Comparative Preparation Example 2 was used instead of the protective agent provided in Preparation Example 9, with the other conditions being the same, so as to obtain an intestinal microbiota product.

### Comparative Example 4

This comparative example adopted the method provided in Example 2 to prepare an intestinal microbiota product, except that in the step S3, an equal mass of the protective agent provided in Comparative Preparation Example 3 was used instead of the protective agent provided in Preparation Example 9, with the other conditions being the same, so as to obtain an intestinal microbiota product.

### Example 13

This example was used for illustrating the preparation of a capsule, which specifically included:
step S1. 79 parts by mass of anhydrous ethanol and 10 parts by mass of beeswax were taken and heated in a water bath kettle at 65° C. until dissolved, then added with 3 parts by mass of food-grade mineral oil, 5 parts by mass of soybean oil and 3 parts by mass of soybean lecithin, mixed uniformly, and subjected to ultrasonication at 65° C. for 1 h to obtain a hydrophobic coating material;
step S2. A capsule shell was taken and completely immersed in the hydrophobic coating material, and allowed to stand for 1 min, and the capsule shell was taken out and repeatedly turned over to remove the excess hydrophobic coating material, and dried in an oven with hot air at 37° C. for 60 min to obtain a hydrophobic capsule shell; and
step S3. 50 g of the intestinal microbiota product provided in Example 1 was taken and filled into the hydrophobic capsule shell to assemble a capsule.

### Example 14

This example was used for illustrating the preparation of a capsule, which specifically included: 50 g of the intestinal microbiota product provided in Example 1 was taken and filled into a capsule shell that was not dip-coated with the hydrophobic coating material to assemble a capsule. The capsule shell used in this example was the same as that in Example 13.

### Test Example

This test example was used for illustrating the relevant performances of the intestinal microbiota product provided in Examples 1-12 and Comparative Examples 1-4 and the capsules provided in Examples 13 and 14, specifically:
1. Deodorizing effect: The odor of intestinal microbiota product provided in Examples 1-12 and Comparative Examples 1-4 was evaluated by ten experimenters, who scored the sensory degrees of the odor in the prepared liquid enteric-coated capsules without knowing the control group and the experimental group (grade A: no odor; grade B: slight odor; grade C: mild odor; grade D: medium odor; grade E: severe odor), and the results were shown in Table 2.

TABLE 2

| Groups | Sensory degree score |
|---|---|
| Example 1 | Grade A |
| Example 2 | Grade A |
| Example 3 | Grade A |
| Example 4 | Grade A |
| Example 5 | Grade A |
| Example 6 | Grade A |
| Example 7 | Grade B |
| Example 8 | Grade A |
| Example 9 | Grade B |
| Example 10 | Grade A |
| Example 11 | Grade B |
| Example 12 | Grade A |
| Comparative | Grade E |
| Example 1 | |
| Comparative | Grade B |
| Example 2 | |
| Comparative | Grade A |
| Example 3 | |
| Comparative | Grade A |
| Example 4 | |

It could be seen from the test results shown in Table 2 that the use of the eluent provided by this invention to treat the feces of the donor could effectively remove the odor of the intestinal microbiota product.
2. The intestinal microbiota product or capsules were placed in a refrigerator at −80°° C. for storage, and sampled at 0 d, 7 d, 30 d and 360 d of storage to test microbiota activity and microbiota taxonomic abundance, and the amount of bacterial yield per unit mass was obtained by calculation;
(1) microbiota activity: Thawed samples from each group were respectively taken and diluted by 100 times with then physiological saline, stained with LIVE/DEADTMBacLightTMBacterial Viability Kit dye for microorganisms in the samples for 15 min, and detected for microbiota activity using a BD Accuri C6 flow cytometer, and the activity changes of the samples before and after treatment were analyzed using BD Accuri™ C6 Plus Software. The test results were as shown in Table 3.

TABLE 3

| | 0 d | | | 7 d | 30 d | 360 d |
|---|---|---|---|---|---|---|
| | microbiota | Amount of bacterial | Mass of | microbiota | microbiota | microbiota |
| | activity | yield per unit mass | microbiota | activity | activity | activity |
| Groups | (%) | (×10¹⁰ cells/g) | pellet (g) | (%) | (%) | (%) |
| Example 1 | 70.2 | 8.71 | 60.3 | 59.7 | 55.7 | 53.1 |
| Example 2 | 77.5 | 14.20 | 79.4 | 73.3 | 70.4 | 69.3 |
| Example 3 | 78.5 | 17.35 | 79.8 | 72.9 | 60.1 | 55.7 |
| Example 4 | 77.8 | 16.31 | 78.2 | 75.4 | 66.3 | 59.8 |
| Example 5 | 78.2 | 14.73 | 75.5 | 73.6 | 67.8 | 59.6 |
| Example 6 | 71.7 | 10.42 | 74.7 | 70.2 | 68.5 | 63.4 |
| Example 7 | 73.6 | 11.67 | 73.6 | 71.8 | 69.2 | 65.5 |
| Example 8 | 71.9 | 11.85 | 78.1 | 69.5 | 66.6 | 61.9 |
| Example 9 | 72.1 | 10.92 | 71.9 | 70.3 | 69.9 | 69.2 |
| Example 10 | 75.1 | 11.89 | 75.0 | 69.3 | 67.6 | 63.1 |
| Example 11 | 75.5 | 11.48 | 78.9 | 68.7 | 67.8 | 60.9 |
| Example 12 | 69.8 | 9.15 | 72.7 | 65.9 | 63.1 | 58.1 |
| Example 13 | 70.2 | 8.62 | 60.1 | 66.0 | 63.1 | 62.8 |
| Example 14 | 70.2 | 8.68 | 60.2 | 61.4 | 56.2 | 53.7 |
| Comparative | 61.4 | 6.56 | 70.2 | 48.1 | 46.3 | 38.3 |
| Example 1 | | | | | | |
| Comparative | 45.6 | 9.61 | 70.1 | 30.6 | 25.7 | 19.5 |
| Example 2 | | | | | | |
| Comparative | 72.3 | 12.23 | 74.8 | 52.5 | 38.5 | 31.1 |
| Example 3 | | | | | | |
| Comparative | 71.3 | 12.45 | 72.6 | 53.8 | 41.7 | 28.9 |
| Example 4 | | | | | | |

It could be seen from the test results shown in Table 3 that, compared with Comparative Examples 1-4, the intestinal microbiota product prepared by the methods provided in Examples 1-12 of this invention each had high microbiota activity greater than 69.8%; and after 360 days of cryopreservation at −80° C., the microbiota activity of the intestinal microbiota product was still higher than 53.1%, and thus the intestinal microbiota product had superior storage stability; at the same time, the introduction of the hydrophobic capsule shell was conducive to further isolating the air, providing a good anaerobic storage environment for the intestinal microbiota product, and further achieving the effect of improving storage stability.
(2) Gastric acid resistance: Artificial gastric fluid was formulated according to the Chinese Pharmacopoeia, preheated to 37° C., added with the capsules provided in Examples 13 and 14, subjected to simulated gastric juice digestion treatment under conditions of 37°° C. and 480 rpm for 2 h, taken out, and tested for microbiota activity according to the test method of (1). The results were as shown in Table 4.

TABLE 4

| Groups | microbiota activity(%) |
|---|---|
| Example 13 | 67.5 |
| Example 14 | 52.3 |

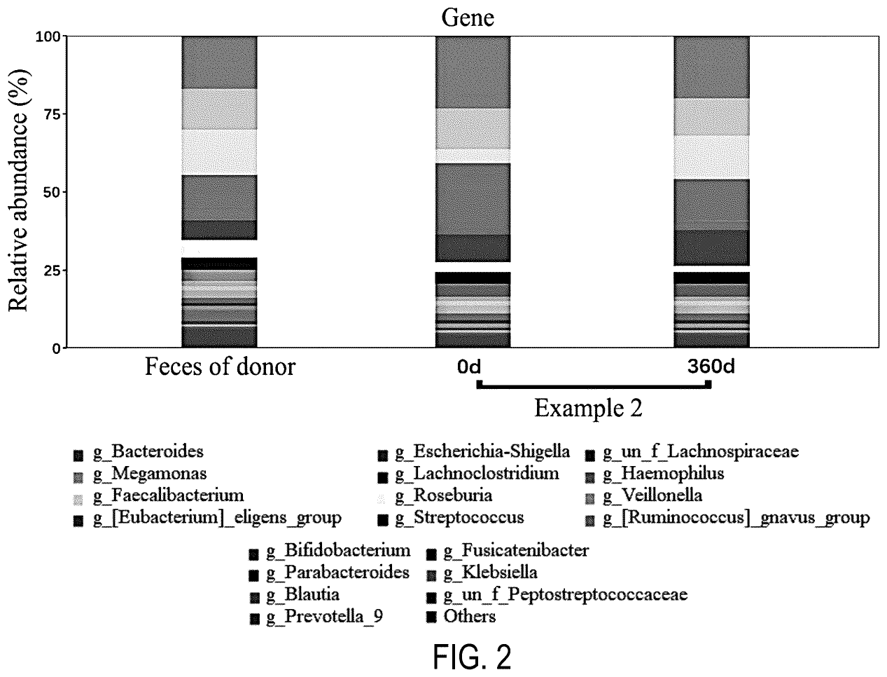

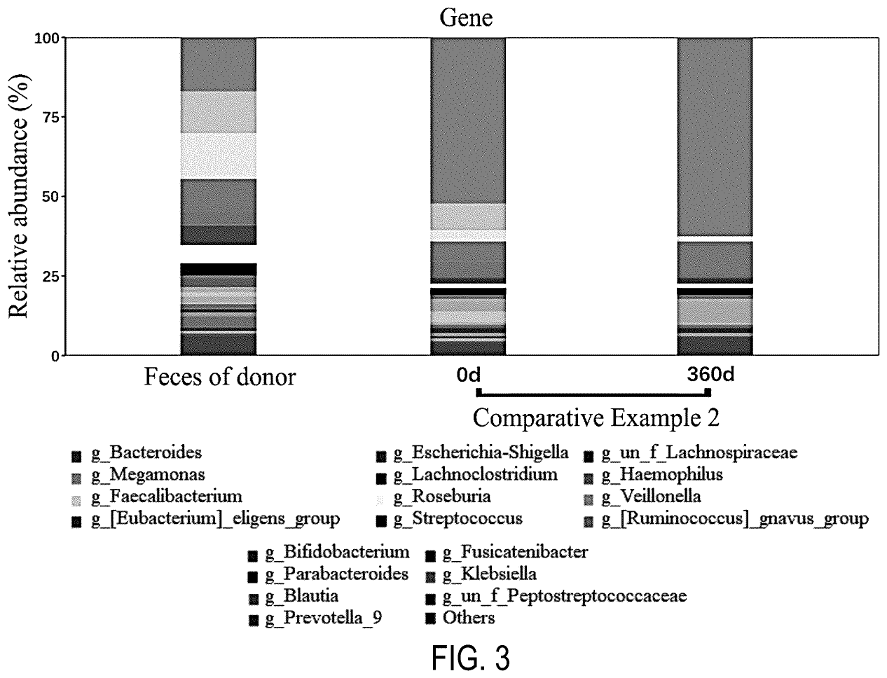

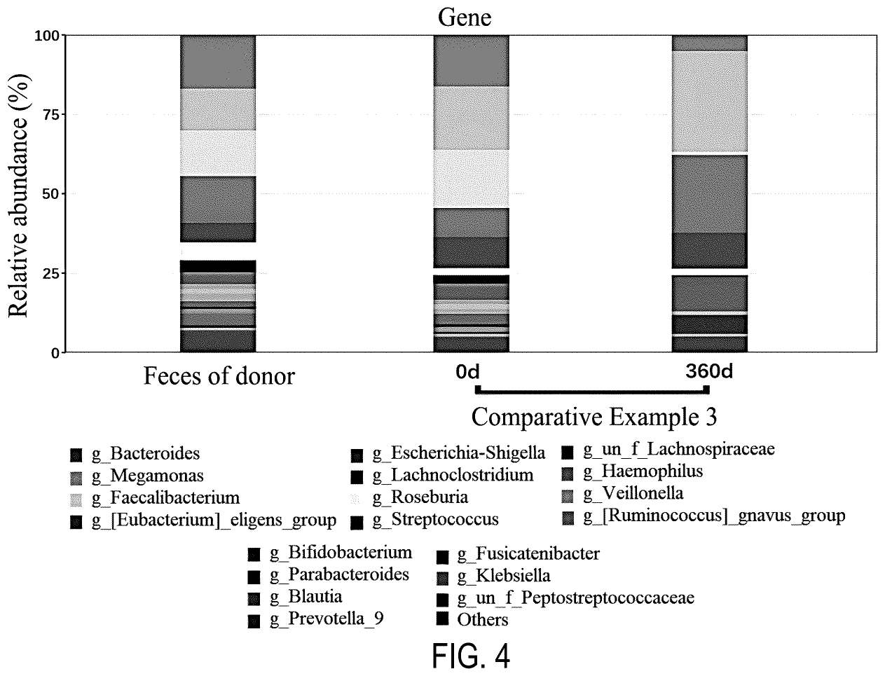

It could be seen from the test results shown in Table 4 that, compared with adopting the ordinary capsule shell in Example 14, the hydrophobic capsule shell provided in Example 13 of this invention had better resistance to gastric acid digestion, had a better protective effect on the intestinal microbiota product, was conducive to the colonization of microbiota in the intestine, and achieved a better effect of precise intestinal microbiota transplantation.
(3) The method for testing microbiota taxonomic abundance was: 0.25 g of the feces of the donor and the intestinal microbiota product of the same donor prepared by the methods provided in Example 2 and Comparative Examples 2 and 3 were respectively taken, and extracted for the total DNA of the intestinal microbiota product using the QIAamp Fast DNA Stool Mini Kit (QIAGEN) according to the instructions; and the extracted total DNA sample was purified, subjected to PCR amplification for the V4 variable region (515F-806R) in the 16S rRNA of the total DNA sample (the reaction system and primer system used in the PCR amplification process were the same and were conventionally used in the art), to construct a qualified sequencing library. Paired-End 150 bp (PE150) sequencing was performed using a MiniSeq platform available from Illumina. After the sequencing was completed, the sequences obtained by sequencing were spliced using flas, and then the primers were removed from the spliced sequences using cutadapt, and the sequences with poor qualities were removed. The spliced sequences were subjected to chimera removal using usearch, and subjected to OTUs clustering based on a similarity of 97%. Based on the results of OTUs clustering analysis, the representative sequences of OTUs were classified by utilizing Silva 132 database, R v3.4.1, GraphPad Prism and SPSS software and using a RDP classifier, and the taxonomic abundance, diversity and evenness indicators of the microbial community were calculated. It should be noted that all the obtained data was expressed as mean±standard deviation (SD). The results were as shown in FIGS. 2-4.

It could be seen from the test results shown in FIGS. 2-4 that, compared with Comparative Examples 2 and 3, the microbiota taxonomic abundance in the intestinal microbiota product prepared by the method provided in Example 2 of this invention had a higher similarity with that in the feces of the donor, and the use of the intestinal microbiota product was conducive to the realization of precise intestinal microbiota transplantation; and after 360 days of cryopreservation at −80° C., the microbiota in the intestinal microbiota product still had high similarity with that in the feces of the donor, indicating that after long-term cryopreservation, the microbiota composition of the intestinal microbiota article changed little and the intestinal microbiota article had superior storage stability.

Although the embodiments of this invention have been shown and described above, it can be understood that the aforementioned embodiments are illustrative and are not to be construed as limitations on this invention. Changes, modifications, substitutions and variations can be made to the aforementioned embodiments within the scope of this invention by those of ordinary skills in the art, without departing from the principle and spirit of this invention.

What is claimed is:

1. A composition for extracting a fecal microbiota, comprising:
an eluent comprising a plant extract and Magnolia officinalis powder in a mass ratio of (0.1-50):(0.001-10), wherein the plant extract is selected from one or more of a Rubus suavissimus extract, a fresh Citrus reticulata extract, a Cymbopogon citratus extract, a clove extract, a bay leaf extract, a charred leaven extract, a licorice extract, a scorched hawthorn fruit extract, an aged Citrus reticulata extract and a scorched barley sprout extract;
a protective agent comprising a small-molecular polyhydroxy compound, a macromolecular multipolymer, an amine substance, a natural emulsifier and an optional carbonate in a mass ratio of (5-50):(1-55):(1-35):(1-30):(0-15), wherein the small-molecular polyhydroxy compound is selected from one or more of mannitol, maltitol, xylitol, trehalose, stachyose, isomaltulose, manno-oligosaccharide, tea polyphenol, vitamin C and malic acid; the macromolecular multipolymer is selected from one or more of fucoidan, konjac manan, corn starch, ethyl cellulose, acetate starch and agar powder; the amine substance is selected from one or more of a fish collagen peptide, glutathione, polyacrylamide, N-acetyl-D-glucosamine and taurine; the natural emulsifier is selected from one or more of arabic gum, carrageenan, gellan gum, guar gum, xanthan gum and medium-chain triglycerides; and the carbonate is potassium carbonate and/or sodium carbonate; and
an optional separation aid comprising a macroporous resin, a functional resin, and a non-resin adsorbent in a mass ratio of (2-5):(2-6):(2-10).

2. The composition for extracting a fecal microbiota according to claim 1, wherein a preparation method of the plant extract specifically comprises: taking and subjecting a plant raw material to water extraction treatment to obtain the plant extract, wherein the plant raw material is selected from one or more of a Rubus suavissimus, a fresh Citrus reticulata, a Cymbopogon citratus, a clove, a bay leaf, a charred leaven, a liquorice root, a scorched hawthorn fruit, an aged Citrus reticulata and a scorched barley sprout.

3. The composition for extracting a fecal microbiota according to claim 2, wherein a method for the water extraction treatment is hot-water leaching and/or ultrasonication.

4. The composition for extracting a fecal microbiota according to claim 2, wherein maltodextrin is added in the water extraction treatment, and a input mass ratio of the plant raw material to the maltodextrin is 1:(1-10).

5. The composition for extracting a fecal microbiota according to claim 1, wherein the plant extract comprises the Rubus Suavissimus extract, the fresh Citrus reticulata extract, the Cymbopogon citratus extract, the clove extract and the scorched barley sprout extract in a mass ratio of (5-10):(0.1-5):(1-3):(0.1-2):(0.1-2).

6. The composition for extracting a fecal microbiota according to claim 1, wherein the eluent further comprises a bicarbonate that is selected from sodium bicarbonate and/or potassium bicarbonate.

7. The composition for extracting a fecal microbiota according to claim 1, wherein the protective agent is selected from one or more of the following formulas:
formula 1: based on a total mass of the protective agent, the protective agent comprises 5-20 wt % of the trehalose, 10-30 wt % of the fucoidan, 15-35 wt % of the fish collagen peptides, 1-10 wt % of the vitamin C, 1-5 wt % of the malic acid and 5-20 wt % of the xanthan gum;
formula 2: based on a total mass of the protective agent, the protective agent comprises 5-20 wt % of the mannitol, 0.1-30 wt % of the arabic gum, 2-10 wt % of the glutathione, 5-30 wt % of the maltitol, 1-30 wt % of the ethyl cellulose and 1-10 w1% of the carrageenan;
formula 3: based on a total mass of the protective agent, the protective agent comprises 20-40 wt % of the corn starch, 5-15 wt9% of the stachyose, 10-25 w1% of the xylitol, 1-15 wt % of the polyacrylamide, 5-15 wt % of the konjac mannan and 1-10 w1% of the gellan gum; and
formula 4: based on a total mass of the protective agent, the protective agent comprises 5-20 wt % of the tea polyphenol, 2-10 wt % of the potassium carbonate, 10-50 wt % of the acetate starch, 2-25 w1% of the N-acetyl-D-glucosamine, 5-30 w1% of the a medium-chain triglyceride and 1-5 wt % of the taurine.

8. The composition for extracting a fecal microbiota according to claim 1, wherein the macroporous resin is selected from one or more of epoxy resin, polyester resin, polyvinyl chloride resin, polyethylene resin, polypropylene resin, phenolic resin, polyurethane resin, polystyrene resin, acrylic resin, ether-ketone resin, urea-formaldehyde resin and ketone-formaldehyde resin.

9. The composition for extracting a fecal microbiota according to claim 1, wherein the functional resin is selected from ion-exchange resin and/or chelating resin.

10. The composition for extracting a fecal microbiota according to claim 1, wherein the non-resin adsorbent is selected from one or more of perlite, diatomaceous earth, activated clay, cellulose, titanium dioxide, activated carbon, coconut shell powder and maifan stone.

11. The composition for extracting a fecal microbiota according to claim 1, wherein an average pore diameter of the macroporous resin is 0.4-1.25 mm, an average particle size of the functional resin is 0.5-1 mm, and an average particle size of the non-resin adsorbent is 0.3-1.2 mm.

12. A method for preparing an intestinal microbiota product using the composition according to claim 1, comprising:
step S1: mixing donor feces with the eluent and optional separation aid, and sequentially subjecting to stirring treatment and filtration treatment to obtain a crude microbiota pellet liquid;
step S2: taking the crude microbiota pellet liquid and sequentially subjecting to tangential flow filtration treatment and centrifugation treatment to obtain a microbiota pellet; and
step S3: taking and mixing the microbiota pellet with the protective agent to obtain the intestinal microbiota product.

13. The method for preparing an intestinal microbiota product according to claim 12, wherein in the step S1, a mixing mass ratio of the feces of the donor, the eluent and the separation aid is (5-15):(50-75):(1-3).

14. The method for preparing an intestinal microbiota product according to claim 12, wherein in the step S1, the stirring treatment is conducted at a rotation speed of 800-1,500 rpm for a time of 4-15 min; and a pore diameter of a filter membrane in the filtration treatment is 20-2,000 μm.

15. The method for preparing an intestinal microbiota product according to claim 12, wherein in the step S2, for the tangential flow filtration treatment, a pore diameter of a filter membrane is 20-2,000 μm, an inlet flow rate is 4-6 L/min/m², an inlet pressure difference is 0.9-1.75 bar, a reflux pressure difference is 0.05-0.2 bar, a transmembrane pressure is 0.5-0.9 bar, and an average membrane flux is 5.1-24.23 L/(m²·h; and the centrifugal treatment is conducted at a rotation speed of 1,000-20,000 g for a time of 2-15 min.

16. The method for preparing an intestinal microbiota product according to claim 12, wherein in the step S3, a mixing mass ratio of the microbiota pellet to the protective agent is (5-20):(1-2).

17. An intestinal microbiota product prepared by the method for preparing an intestinal microbiota product according to claim 12.

18. A method for preparing a capsule, comprising: taking a hydrophobic coating material to conduct coating on a capsule shell to obtain a hydrophobic capsule shell; and taking the hydrophobic capsule shell to package the intestinal microbiota product according to claim 17 to obtain the capsule;
wherein the hydrophobic coating material comprises beeswax, mineral oil, vegetable oil and an emulsifier at a mass ratio of (5-10):(1-3):(3-5):(1-3).

19. A capsule prepared by the method for preparing a capsule according to claim 18.

20. A use of the intestinal microbiota product according to claim 17 in precise microbiota transplantation.
